(19) United States
(12) Patent Application Publication
(10) Pub. No.: US 2017/0112454 A1
(43) Pub. Date: Apr. 27, 2017

(54) COMPUTED TOMOGRAPHY APPARATUS

(71) Applicant: SAMSUNG ELECTRONICS CO., LTD., Suwon-si (KR)

(72) Inventors: Alexander YUN, Seoul (KR); Toshihiro RIFU, Suwon (KR); IL SEONG, Yongin (KR)

(73) Assignee: SAMSUNG ELECTRONICS CO., LTD., Suwon-si (KR)

(21) Appl. No.: 15/238,134

(22) Filed: Aug. 16, 2016

(30) Foreign Application Priority Data
Oct. 23, 2015 (KR) 10-2015-0148339

(51) Int. Cl. A61B 6/03; A61B 6/04; A61B 6/00

(52) U.S. Cl. CPC A61B 6/032; A61B 6/4435; A61B 6/0407; A61B 6/54; A61B 6/4035; A61B 6/461; A61B 6/467

(57) ABSTRACT

A computed tomography apparatus including a gantry rotated along a rail in the shape of a ring is provided. The computed tomography apparatus according to an embodiment includes an x-ray source to radiate x-rays, an x-ray detector to detect the x-rays, a gantry at which the x-ray source and the x-ray detector are mounted, and a rail provided in the shape corresponding to an outer side surface of the gantry, and the gantry is configured to rotate along an inner side surface of the rail.

### CROSS-REFERENCE TO RELATED APPLICATION

This application claims the priority benefit of Korean Patent Application No. 10-2015-0148339, filed on Oct. 23, 2015, in the Korean Intellectual Property Office, the disclosure of which is incorporated herein by reference.

### BACKGROUND

1. Field

Embodiments of the present disclosure relate to a computed tomography apparatus having an improved rotational structure.

2. Description of the Related Art

A medical imaging apparatus is an apparatus configured to obtain an inside structure of a subject in the form of an image. The medical imaging apparatus is referred to as a non-invasive apparatus, and is provided to show a user with detailed structural information, internal tissues, and flow of fluid at an inside of a body by photographing and processing such. The user such as a physician may be able to diagnose the health status and disease of a patient by use of the medical images being output from the medical imaging apparatus.

As for the medical imaging apparatus configured to photograph the subject by radiating x-rays at the patient, a Computed Tomography (CT) is typically present. The Computed Tomography as such is capable of reconstructing images by use of a computer by penetrating the x-rays from a number of directions, and since the penetrability of the x-rays may be different by the tissue structuring the subject, an inside structure of the subject may be able to be visualized in the form of images by use of attenuation coefficient, which is expressed in numeric figures.

The Computed Tomography (CT) is capable of providing tomographic images with respect to the subject, and when compared to an x-ray apparatus in general, is capable of expressing the inside structure of the subject, such as a kidney, lungs, or any other organ, without overlapping, and thus is widely used as to provide precise diagnosis of a disease.

### SUMMARY

Therefore, it is an aspect of the present disclosure to provide a computed tomography apparatus provided with a gantry configured to rotate along a rail having the shape of a ring.

Additional aspects of the disclosure will be set forth in part in the description which follows and, in part, will be obvious from the description, or may be learned by practice of the disclosure.

In accordance with an aspect of the present disclosure, a computed tomography apparatus includes an x-ray source to radiate x-rays; an x-ray detector to detect the x-rays; a gantry at which the x-ray source and the x-ray detector are mounted; and a rail provided in the shape corresponding to an outer side surface of the gantry, and the gantry is configured to rotate along an inner side surface of the rail.

A plurality of driving wheels may be provided in between the inner side surface of the rail and the outer side surface of the gantry.

The driving wheel may be mounted at the outer side surface of the gantry.

The driving wheel may be provided to move along the rail based on a driving force received from a driving source.

The rail may be provided in the shape of a ring.

The computed tomography apparatus may further include a housing forming an exterior appearance of the computed tomography apparatus, and the rail may be mounted at the housing.

The computed tomography apparatus may further include a housing forming an exterior appearance of the computed tomography apparatus, and the rail may be formed at an inner side surface of the housing.

The rail may be provided with a space formed thereto to which at least a portion of the gantry is inserted.

The rail may be provided in a single unit.

The gantry may be provided as to be rotated while magnetically levitated.

The outer side surface of the gantry may be provided with a first magnetic unit, and the inner side surface of the rail may be provided with a second magnetic unit.

The x-ray source and the x-ray detector may be provided to face each other.

In accordance with another aspect of the present disclosure, a computed tomography apparatus includes a gantry having the shape of a cylinder at which an x-ray source and an x-ray detector are mounted to face each other, and rotatably provided; a rail extended along an outer circumference of the gantry, and provided to guide the rotation of the gantry; and a driving wheel provided in between the gantry and the rail.

The driving wheel may be provided to be rotated based on a driving force received from an outside driving source.

The driving wheel may be provided in a plurality of units, and may be mounted at an outer side surface of the gantry.

The rail may be provided in a single unit along an outer circumferential surface of the gantry.

In accordance with still another aspect of the present disclosure, a computed tomography apparatus includes a gantry at which an x-ray source and an x-ray detector are mounted; and a rail extended while corresponding to an outer circumferential surface of the gantry, and the gantry is provided to be rotated while magnetically levitated with respect to the rail.

The gantry may be provided in the shape of a cylinder, and the rail may be provided in the shape of a ring while wrapping around at least a portion of the gantry.

The x-ray source and the x-ray detector may be positioned as to face each other.

The rail may be provided in a single unit.

In accordance with still another aspect of the present disclosure, a computed tomography apparatus includes a gantry at which an x-ray source and an x-ray detector are mounted to face each other; a driving wheel mounted at an outer circumferential surface of the gantry; and a rail extended along the circumference of an outer side of the gantry, and provided as to have the driving wheel drive.

The rail is provided in a single unit.

As is apparent from the above, the computed tomography apparatus in accordance with an embodiment of the present disclosure can be reduced in the volume and weight by improving the rotational structure of a gantry.

### BRIEF DESCRIPTION OF THE DRAWINGS

These and/or other aspects of the disclosure will become apparent and more readily appreciated from the following description of the embodiments, taken in conjunction with the accompanying drawings.

FIG. 1 is a drawing illustrating a computed tomography apparatus according to an embodiment of the present disclosure.

FIG. 2 is a drawing illustrating an image of a subject positioned on a table according to an embodiment of the present disclosure.

FIG. 3 and FIG. 4 are drawings illustrating the computed tomography apparatus according to an embodiment of the present disclosure photographing the subject.

FIG. 5, FIG. 6A, and FIG. 6B are drawings illustrating a gantry and a rail according to an embodiment of the present disclosure.

FIG. 7 and FIG. 8 are drawings illustrating a gantry and a rail according to another embodiment of the present disclosure.

FIG. 9 is a drawing illustrating an air flow passing through the gantry according to an embodiment of the present disclosure.

FIG. 10 is a drawing illustrating components being assembled at the gantry according to an embodiment of the present disclosure.

### DETAILED DESCRIPTION

Reference will now be made in detail to the embodiments of the present disclosure, examples of which are illustrated in the accompanying drawings, wherein like reference numerals refer to like elements throughout.

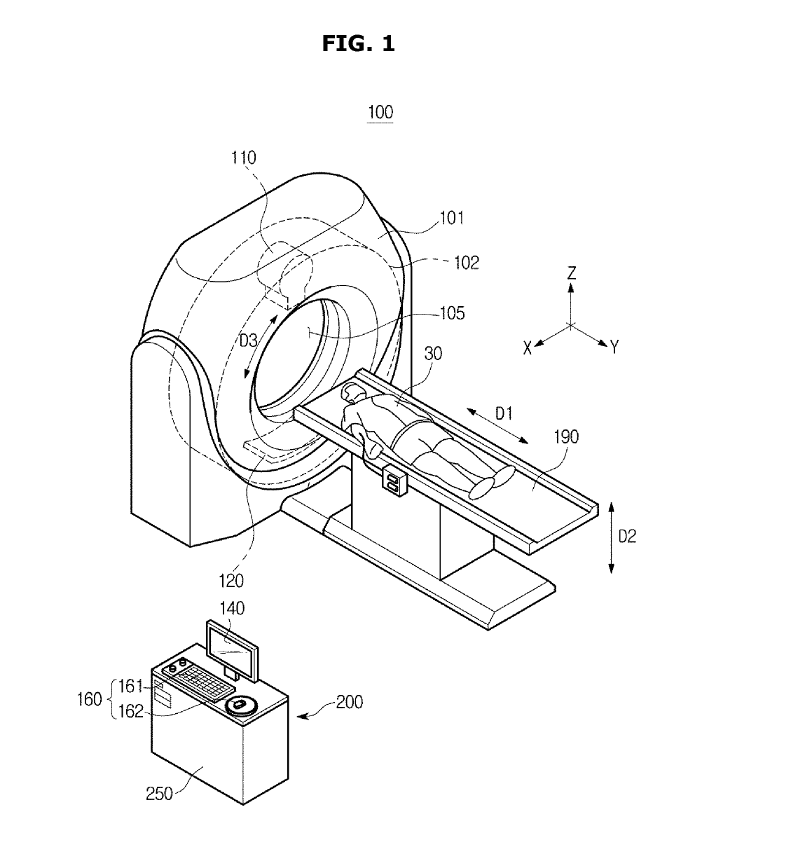

FIG. 1 is a drawing illustrating a computed tomography apparatus according to an embodiment of the present disclosure.

Referring to FIG. 1, a computed tomography apparatus 100 according to an embodiment of the present disclosure may include a table 190 at which a body and a subject are positioned. The body may include a housing 101 and a gantry 102. The computed tomography apparatus 100 is provided with motions thereof controlled by use of a control apparatus 200.

A subject 30 may be, for example, a body of a human, a body of an animal, or a tissue at an inside of a body such as vein, bone, or muscle, but the type of the subject 30 is not limited hereto. The subject 30 may be referred to any substance as long as at some of an inside structure the substance may be visualized in the form of an image by use of the computed tomography apparatus 100.

An inside of the housing 101 may be provided with the gantry 102 having the shape of a cylinder mounted thereto. An inside of the gantry 102 may be provided with an x-ray source 110 to radiate x-rays and an x-ray detector 120, for example, facing the x-ray source to detect the x-rays.

The housing 101 may be provided in the approximate shape of a cylinder to correspond to the shape of the gantry 102. An empty space in the middle of the housing 101 and the gantry 102 may be referred to as a bore 105. The subject may be positioned at the bore 105 such that a computed tomographic photographing may be performed.

The x-ray source 110 is an apparatus configured to generate x-rays and radiate the x-rays at the subject 30. The gantry 102 may be provided with a filtering unit to filter the x-rays generated from the x-ray source 110.

The x-ray detector 120 is an apparatus configured to detect the x-rays penetrated through the subject 30. The x-ray detector 120 may be provided at an opposite side of the x-ray source 110. The subject 30 may be positioned in between the x-ray source 110 and the x-ray detector 120. The x-rays generated from the x-ray source 110 may be detected through the x-ray detector 120 after passing through the subject 30.

The gantry 102 may rotate around the bore 105. The x-ray source 110 and the x-ray detector 120 may be rotated together with the gantry 102. The x-ray source 110 and the x-ray detector 120 may detect x-ray images with respect to the subject 30 while rotating around the subject 30.

The table 190 may be provided with the subject 30 positioned on the table 190. The subject 30 may be transferred to an inside of the bore 105 while positioned on the table 190. The table 190 may be moved in a y-axis direction and in a z-axis direction while maintaining a horizontal state with respect to a ground level. The direction in which the table 190 is moved toward the y-axis is referred to as a D1 direction, and the direction in which the table 190 is moved toward the z-axis is referred to as a D2 direction. The table 190 may be moved while moving in the D1 and D2 directions such that the diagnosis portion to be photographed may be positioned in between the x-ray source 110 and the x-ray detector 120. The table 190 may also be movably provided in an x-axis direction as to adjust the left and right gaps at an inside of the bore 150.

The control apparatus 200 may include a controlling body 250, a manipulating unit 160 configured to be manipulated by a user, and a display unit 140 at which the information related to the motions of the computed tomography apparatus 100 may be displayed.

A user may input a command through the manipulating unit 160 to control the motions of the computed tomography apparatus 100. The manipulating unit 160 may include a keyboard 161 and a mouse 162. The manipulating unit 160 may include, other than the keyboard 161, and the mouse 162, for example, a track ball, a foot switch, a foot pedal, and a touch pad.

The display unit 140 may be provided with a variety of information related to the motions of the computed tomography apparatus 100 to be displayed thereto. The display unit 140 may be implemented, for example, as a Cathode Ray Tube (CRT), a Light Emitting Diode (LED), or a Liquid Crystal Display (LCD). The display unit 140 may be provided in the form of a Touch Screen Panel (TCP), and the display unit 140 and the manipulating unit 160 may be integrally implemented. The display unit 140 may be provided in a plurality of units, and may be provided to display different information at the each of the plurality of display units 140.

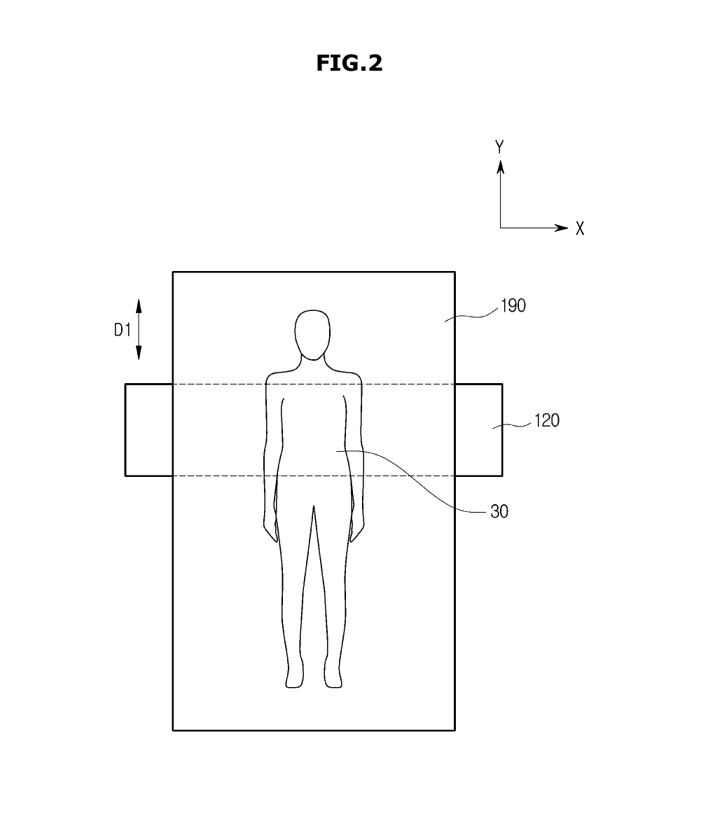

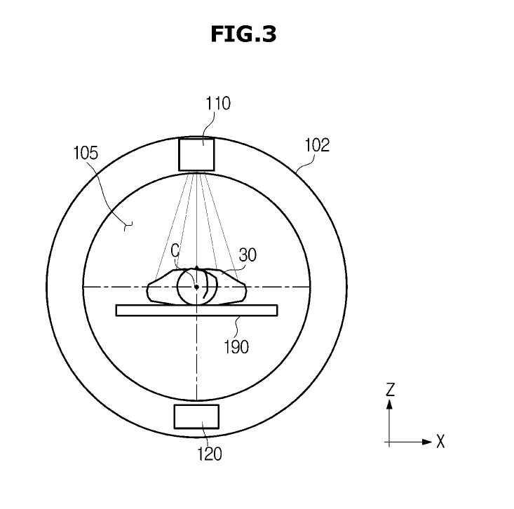

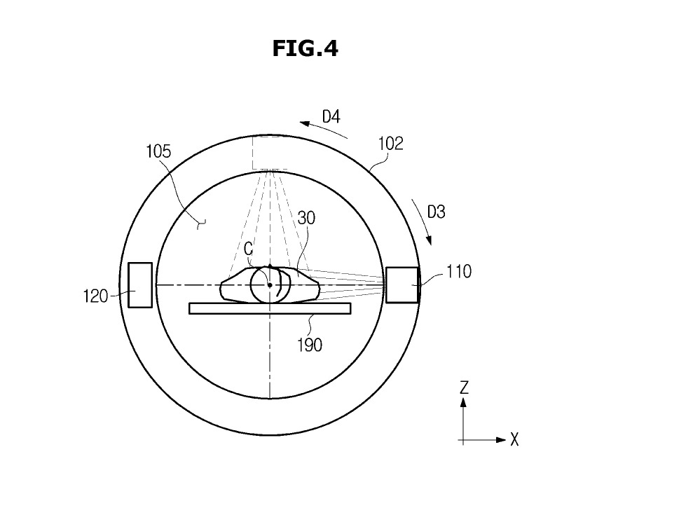

FIG. 2 is a drawing illustrating an image of a subject positioned on a table according to an embodiment of the present disclosure, and FIG. 3 and FIG. 4 are drawings illustrating an image of the computed tomography apparatus according to an embodiment of the present disclosure photographing the subject.

Referring to FIG. 2 to FIG. 4, the table 190 at which the subject 30 is positioned may be positioned at an inside of the bore 105 while moving in the D1 direction. In addition, the table 190 may be moved in left and right directions as well as upward and downward directions so that the central portion of an area of interest of the subject 30 may be in conformity with the central portion of the bore 105.

The gantry 102 at which the x-ray source 110 and the x-ray detector 120 are mounted may perform a tomographic photographing with respect to an area of interest of the subject 30 while the gantry 102 is rotated in the D3 direction (or a reverse D4 direction).

A gantry implemented in a conventional computed tomography apparatus is provided to be rotated while receiving a driving force from a driving apparatus such as a motor provided at one side. As one example, the gantry is provided to be rotated while mounted at a rotor provided at the motor. A stator is provided at an outer circumferential surface of the rotor, and the rotor is rotated at one side of the stator while received with an outside power. A bearing is provided in between the stator and the rotor so that the rotor may be rotated. The rotor at which the gantry may be mounted, as well as the stator corresponding to the rotor, is provided at an inside of a housing. Therefore, the housing is provided with larger volume by the rotor and the stator corresponding to the gantry, and the weight of the body of a computed tomography apparatus is provided with heavier weight by the rotor and the stator provided at an inside of the housing.

According to an exemplary embodiment of the present disclosure, a structure of delivering a rotational force provided with the rotor configured to rotate the gantry may be omitted. By having the rotor omitted, when compared to a conventional practice, the volume and weight of the body of the computed tomography apparatus 100 may be reduced.

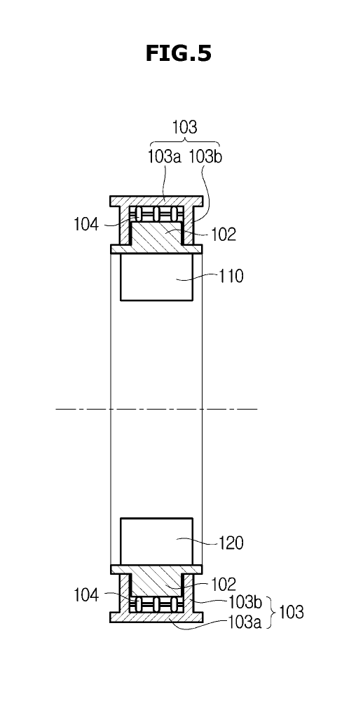

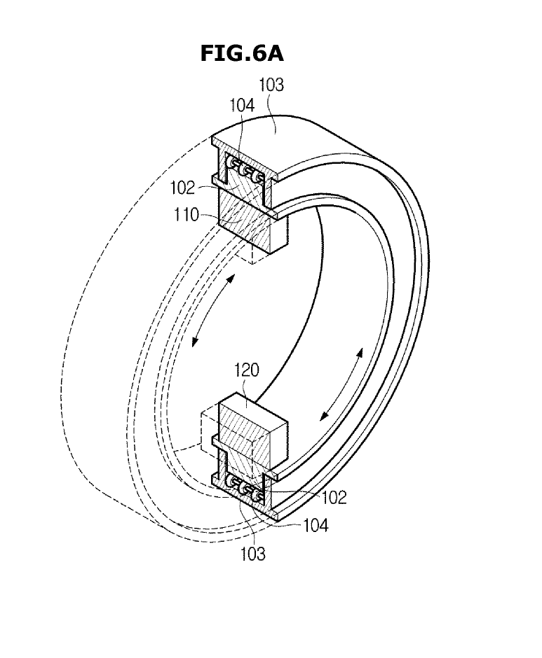

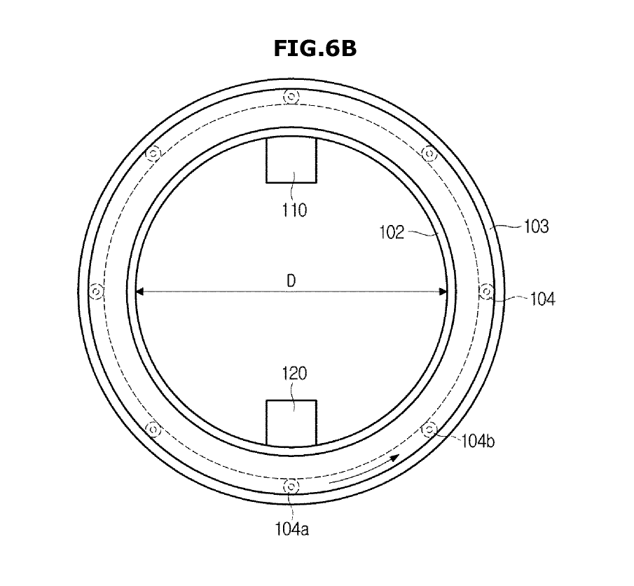

FIG. 5, FIG. 6A and FIG. 6B are drawings illustrating the gantry and a rail according to an embodiment of the present disclosure.

Referring to FIG. 5, FIG. 6A, and FIG. 6B, the gantry 102 according to an embodiment of the present disclosure may be rotatably provided by use of the principle of a monorail. An outer circumferential surface of the gantry 102 is provided with a rail in the shape of a singular ring, and the gantry 102 may be provided to be rotated along the rail. The straight line passing the central portion of the gantry 102 and the straight line passing the central portion of the rail may be coincident to each other.

As one example, an outer side of the gantry 102 may be provided with a rail 103 corresponding to an outer circumferential surface of the gantry 102. The gantry 102 may be rotatably provided along the rail 103.

A driving wheel 104 may be inserted in between the gantry 102 and the rail 103. The driving wheel 104 may be provided in a plurality of units in between the gantry 102 and the rail 103. The driving wheel 104 may be driven along the rail 103 while mounted at the gantry 102. The driving wheel 104 may be rotated while receiving a driving force from an outside driving source. As for another example, the driving wheel 104 may be provided to be rotated while mounted at the rail 103. The driving wheel 104 may rotate the gantry 102 while rotating at an inner side surface of the rail 103.

An embodiment in which the plurality of driving wheels 104 is driven along the rail 103 while mounted at an outer circumferential surface of the gantry 102 is described.

The plurality of driving wheels 104 may be disposed at an inside of a space formed by use of an outer side surface of the gantry 102 and an inner side surface of the rail 103 while being spaced apart from one another. The driving wheel 104 may be driven along the rail 103 while mounted at an outer side surface of the gantry 102. A certain one driving wheel 104a from the plurality of driving wheels 104 may be rotated along with the gantry 102. Therefore, a certain one driving wheel from the plurality of driving wheels 104 may not be continuously positioned at a lower portion of the gantry 102, and may be positioned at a variety of positions such as a left side or an upper side of the gantry 102. As illustrated in FIG. 6B, a first driving wheel 104a positioned at a lower portion of the gantry 102 may be moved to the position of a second driving wheel 104b positioned at a side of the gantry 102 while rotated along the gantry 102.

As a certain one of the driving wheels 104 may be provided to be positioned at a lower portion of the gantry 102 without being continuously positioned at a lower portion of the gantry as the plurality of driving wheels 104 is rotated, the weight of the gantry 102 may be distributed and supported at the plurality of driving wheels 104. Therefore, when compared to a case in which the certain one of the driving wheels 104 is provided to be positioned at a lower portion of the gantry 102, the driving wheel 104 may be prevented from being damaged.

The rail 103 may include a first rail unit 103a extended along an outer side circumference of the gantry 102, and a second rail unit 103b extended from an inner side surface of the first rail unit 103a. At least a portion of the gantry 102 may be rotated along the rail 103 while inserted into a space at which the first rail unit 103a and the second rail unit 103b are formed.

An embodiment in which the portion of the gentry 102 is inserted into the space at which the rail 103 is formed is described above, but the structure in which the gantry 102 is provided to be rotated along the rail 103 is not limited to the above-provided disclosure. For example, a portion of the rail 103 may be inserted into a space formed at an outer side surface of the gantry 102.

The rail 103 may be provided at an inner side surface of the housing 101. The rail 103 may be mounted at an inner side surface of the housing 101 while separately provided, or the shape of an inner side surface of the housing 101 may be formed together with the rail 103.

Conventionally, the gantry 102 is rotated while mounted at a rotor, but according to an exemplary embodiment of present disclosure, the gantry 102 may be provided to be rotated along the rail 103 extended along an outer side circumference of the gantry 102, and the rotor at which the conventional gantry 102 is mounted may be omitted. By omitting the rotor, the volume and weight of the body of the computed tomography apparatus 100 may be reduced when compared to a conventional computed tomography apparatus having the rotor.

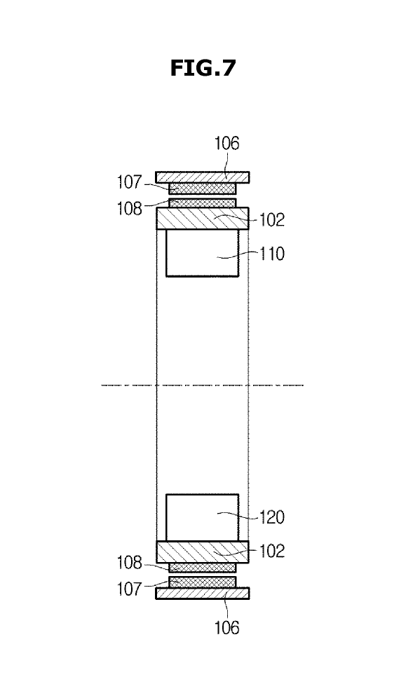

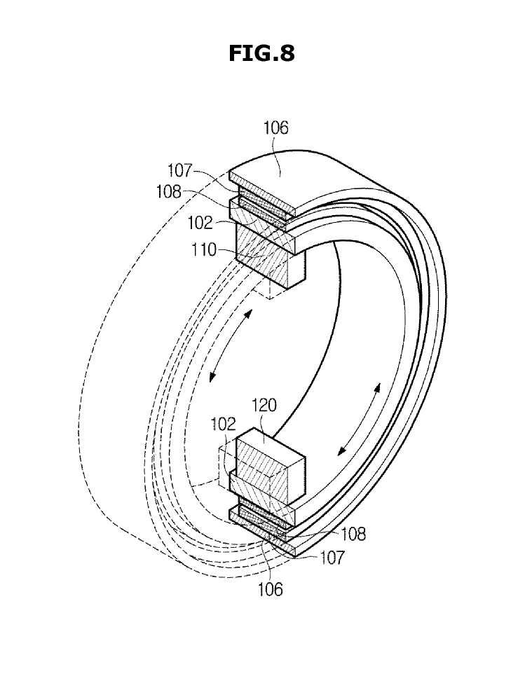

FIG. 7 and FIG. 8 are drawings illustrating a gantry and a rail according to another embodiment of the present disclosure.

Referring to FIG. 7 and FIG. 8, the gantry 102 according to another embodiment of the present disclosure may be rotatably provided by use of a principle of magnetic levitation using magnetic fields. The magnetic levitation refers to a suspending of an object by use of an electromagnetic force.

An outer circumferential surface of the gantry 102 may be provided with a rail 106 having the shape of a ring. The gantry 102 may be provided as to rotate along the rail 106. An outer circumferential surface of the rail 106 may be provided with a first magnetic unit 107, and an inner circumferential surface of the gantry 102 may be provided with a second magnetic unit 108 corresponding to the first magnetic unit 107. As one example, at least one of the first magnetic unit 107 and the second magnetic unit 108 may be provided in the form of electromagnet configured to change polarity according to the direction of current. As another example, a certain one of the first magnetic unit 107 and the second magnetic unit 108 may be provided in the form of a superconductive material.

A gantry 102 may rotate along the rail 106 by use of the principle of magnetic levitation, using a force suspending the gantry 102 from the rail 106 and a force provided in a rotation direction of the gantry 102.

A method of suspending the gantry 102 from the rail 106 may include a repulsive method using a repulsive force of the both polarities of a magnet, and an attractive method using the gravitational force between a magnet and a magnetic material.

As one example, in a case when the gantry 102 is suspended from the rail 106 by use of the repulsive method, the first magnetic unit 107 of the rail 106 is provided with an electromagnet disposed thereto, and the second magnetic unit 108 of the gantry 102 is provided with a plurality of permanent magnets disposed thereto. As the gantry 102 is moved along the rail 106, one side of the second magnetic unit 108 may be provided with a repulsive force, by use of the principle of electromagnetic induction, as the one side of the second magnetic unit 108 is provided with an identical polarity to the polarity of the first magnetic unit 107 of the rail 107. As the other side of the second magnetic unit 108 is provided with a gravitational force as the second magnetic unit 108 is provided with a different polarity from the polarity of the first magnetic unit 107, the repulsive force may be maintained at the first magnetic unit 107 and at the other side of the second magnetic unit 108 if the direction of the current of the first magnetic unit 107 is oppositely changed. By repeating the motion of changing the directions of the current flowing at the first magnetic unit 107, the gantry 102 may be able to remain suspended from the rail 106.

As another example, in a case when the gantry 102 is suspended from the rail 106 by use of the attractive method, the second magnetic unit 108 provided at the gantry 102 may be provided with an electromagnet configured to change polarity according to the direction of current. The rail 106 may be provided with conductive metallic material. When the current is provided to flow at the second magnetic unit 108, an upper portion of the gantry 102, by use of the polarity of the second magnetic unit 108, may tend to be attached to the rail 106 positioned at an upper side. When the current is discontinued prior to the gantry 102 completely attached to the rail 102, the attractive force of the second magnetic unit 108 is not present, and the upper portion of the gantry 192 may descend. If the current is provided to flow again prior to a lower surface of the gantry 102 making contact at the rail 106 after the gantry 102 is descended, a gravitational force may be applied in between the upper portion of the gantry 102 and an upper surface of the rail 106, and a lower portion of the gantry 102 may be suspended from the rail 106. By repeating the motions of continuing and discontinuing the flow of the current at the second magnetic unit 108 provided at the gantry 102, the gantry 102 may be able to remain suspended from the rail 106.

A linear motor may be used as to rotate the gantry 102. An electromagnet may be provided at the gantry 102, and conductive metallic material may be positioned at the rail 106. If the directions of the current flowing at the electromagnet provided at the gantry 102 are changed, the magnetic fields are changed, and thus a thrust force may be generated by use of an electromotive force in between the electromagnet and the conductive metallic material.

The rail 106 may be provided at an inner side surface of the housing 101 while separately provided, and a portion of an inner side surface of the housing 101 may be provided as the rail 106.

As the gantry 102 may be provided to be rotated by use of the principle of magnetic levitation, the conventional rotor conventionally mounted at the gantry 102 may be omitted. By omitting the rotor, the volume and weight of the body of the computed tomography apparatus 100 may be reduced when compared to a conventional computed tomography apparatus having the rotor.

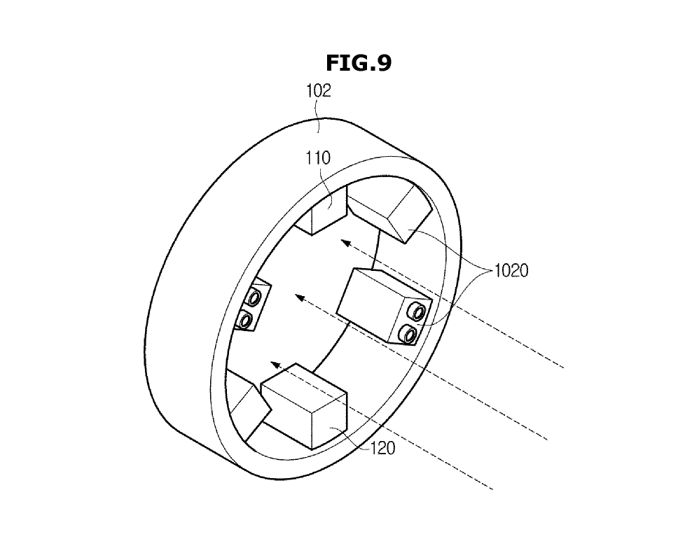

FIG. 9 is a drawing illustrating an air flow passing through the gantry according to an embodiment of the present disclosure.

Referring to FIG. 9, an inner side surface of the gantry 102 according to an embodiment may be provided, not only to the x-ray source 110 and the x-ray detector 120, but also with various electronic components 1020 needed to be provided at the computed tomography apparatus 100. The electronic components 1020 as such may generate a large amount of heat at the time of when the computed tomography apparatus 100 is in motion.

In a conventional case, at least a portion of a rear of an inner side of a gantry is covered by use of the structure of a stator provided to entirely wrap around the gantry and the structure of a rotor provided at an inner side of the stator. The diameters of an opening formed at an inner side of the gantry are provided with a smaller rear side diameter thereof when compared to a front side diameter thereof. In the case as such, the flow of air passing through the gantry is interfered, and the various electronic components provided at the gantry are not efficiently cooled.

However, according to an exemplary embodiment of the present disclosure, an inner side diameter D (FIG. 6B) from each of the front side and the rear side of the gantry 102 may be provided to be identical to an another. Therefore, the air may be easily moved through the bore 105 formed at an inner side of the gantry 102. As the air is easily moved, the electronic components 1020 may be efficiently cooled.

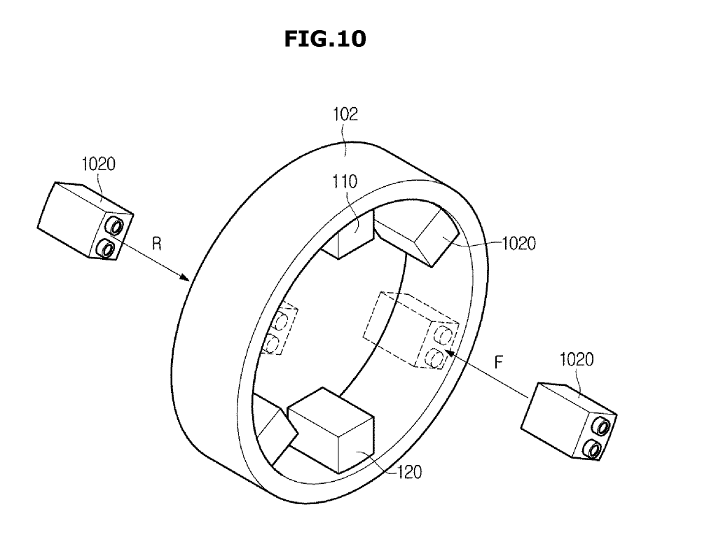

FIG. 10 is a drawing illustrating an image of components being assembled at the gantry according to an embodiment of the present disclosure.

Referring to FIG. 10, the various electronic components 1020 provided at the gantry 102 may be assembled at a front or at a rear of the gantry 102.

In a conventional case, since at least a portion of a rear of an inner side of a gantry is covered by use of the structure of a stator provided to entirely wrap around the gantry and the structure of a rotor provided at an inner side of the stator, the electronic components are difficult to be assembled at the rear of the gantry. Therefore, in the conventional case, the electronic components are mounted at an inner side surface of the gantry from the front of the gantry.

However, according to an exemplary embodiment of the present disclosure, the rear of the gantry 102 is provided with the diameter D that is identical to the diameter D of the front of the gantry 102, and thus an assembly space may be adequately secured to assemble the electronic components 1020 even at a rear R of the gantry 102. Therefore, a user, not only at a front F of the gantry 102, but also at the R of the gantry 102, may assemble the various electronic components 1020 having the x-ray source 110 and the x-ray detector 120 to be mounted at an inner side surface of the gantry 102.

According to exemplary embodiments of the present disclosure, a light-weight, miniaturized computed tomography apparatus 100 may be implemented by improving the structure of rotating the gantry 102.

Although a few embodiments of the present disclosure have been shown and described, it would be appreciated by those skilled in the art that changes may be made in these embodiments without departing from the principles and spirit of the disclosure, the scope of which is defined in the claims and their equivalents.

What is claimed is:

1. A computed tomography apparatus, comprising:
an x-ray source to radiate x-rays;
an x-ray detector to detect the x-rays radiated by the x-ray source;
a gantry at which the x-ray source and the x-ray detector are mounted; and
a rail having a shape corresponding to an outer side surface of the gantry,
wherein the gantry is configured to rotate along an inner side surface of the rail.

2. The computed tomography apparatus of claim 1, wherein:
a plurality of driving wheels in between the inner side surface of the rail and the outer side surface of the gantry.

3. The computed tomography apparatus of claim 2, wherein:
the driving wheel is mounted at the outer side surface of the gantry.

4. The computed tomography apparatus of claim 2, wherein:
the driving wheel is movable along the rail based on a driving force received from a driving source.

5. The computed tomography apparatus of claim 1, wherein:
the rail is in the shape of a ring.

6. The computed tomography apparatus of claim 1, further comprising:
a housing forming an exterior appearance of the computed tomography apparatus,
wherein the rail is mounted at the housing.

7. The computed tomography apparatus of claim 1, further comprising:
a housing forming an exterior appearance of the computed tomography apparatus,
wherein the rail is formed at an inner side surface of the housing.

8. The computed tomography apparatus of claim 1, wherein:
the rail includes a space into which at least a portion of the gantry is inserted.

9. The computed tomography apparatus of claim 1, wherein:
the rail is a single unit.

10. The computed tomography apparatus of claim 1, wherein:
the gantry is configured to rotate while magnetically levitated.

11. The computed tomography apparatus of claim 10, wherein:
the outer side surface of the gantry includes a first magnetic unit, and the inner side surface of the rail includes a second magnetic unit.

12. The computed tomography apparatus of claim 1, wherein:
at least a part of the x-ray source and at least a part of the x-ray detector face each other.

13. A computed tomography apparatus, comprising:
a gantry having the shape of a cylinder at which an x-ray source and an x-ray detector are mounted to face each other, and configured to rotate;
a rail extending along an outer circumference of the gantry to guide the rotation of the gantry; and
a driving wheel in between the gantry and the rail.

14. The computed tomography apparatus of claim 13, wherein:
the driving wheel is rotated based on a driving force received from an outside driving source.

15. The computed tomography apparatus of claim 13, wherein:
the driving wheel is a plurality of units, and mounted at an outer side surface of the gantry.

16. The computed tomography apparatus of claim 13, wherein:
the rail is a single unit along an outer circumferential surface of the gantry.

17. A computed tomography apparatus, comprising:
a gantry at which an x-ray source and an x-ray detector are mounted; and
a rail extending along an outer circumferential surface of the gantry,
wherein the gantry is rotatable while magnetically levitated with respect to the rail.

18. The computed tomography apparatus of claim 17, wherein:
the gantry is in the shape of a cylinder, and the rail is in the shape of a ring and wraps around at least a portion of the gantry.

19. The computed tomography apparatus of claim 17, wherein:
at least a part of the x-ray source and at least a part of the x-ray detector face each other.

20. The computed tomography apparatus of claim 17, wherein:
the rail is a single unit.
